(19) United States
(12) Patent Application Publication
(10) Pub. No.: US 2020/0188642 A1
(43) Pub. Date: Jun. 18, 2020

(54) ELECTROSPUN FIBER-COATED ANGIOPLASTY DEVICES AND METHODS

(71) Applicant: NANOFIBER SOLUTIONS, LLC, Hilliard, OH (US)

(72) Inventors: Kevin J. NELSON, Columbus, OH (US); Jed JOHNSON, London, OH (US)

(73) Assignee: NANOFIBER SOLUTIONS, LLC, Hilliard, OH (US)

(21) Appl. No.: 16/713,272

(22) Filed: Dec. 13, 2019

Related U.S. Application Data
(60) Provisional application No. 62/779,046, filed on Dec. 13, 2018.

(51) Int. Cl. A61M 25/10; A61L 29/16; A61L 29/04

(52) U.S. Cl. CPC A61M 25/104; A61M 25/1036; A61M 2025/1043; A61L 29/048; A61L 29/16

(57) ABSTRACT

An angioplasty device may comprise a catheter, a balloon disposed on the catheter, and a scaffold disposed over the balloon. The scaffold may comprise an electrospun polymer fiber and an agent dispersed within the fiber. A method of making such an angioplasty device may comprise obtaining a catheter having a balloon disposed on it, contracting the balloon, and electrospinning a polymer solution onto the surface of the catheter having the balloon, thereby forming a scaffold disposed over the balloon. The polymer solution may comprise a polymer, a solvent, and an agent. A method of performing an angioplasty procedure may comprise inserting such an angioplasty device into a blood vessel, placing the angioplasty device near a lesion within the blood vessel, expanding the balloon, thereby contacting the lesion with the scaffold, contracting the balloon, and removing the angioplasty device from the blood vessel.

### CROSS-REFERENCE TO RELATED APPLICATIONS

This application claims priority to and benefit of U.S. Provisional Application Ser. No. 62/779,046, filed Dec. 13, 2018, entitled “Electrospun Fiber-Coated Angioplasty Devices and Methods,” which is incorporated herein by reference in its entirety.

### BACKGROUND

Cardiovascular disease is the leading cause of death for Americans. Atherosclerosis, or the narrowing of a blood vessel due to the buildup of plaque from fat or cholesterol deposition, is frequently treated using percutaneous transluminal angioplasty. During an angioplasty procedure, a balloon-tipped catheter is directed through the vessel toward the site of the arterial constriction. After being positioned inside the arterial stenosis, the balloon is expanded for a precise amount of time. This expansion breaks apart and compresses the plaque buildup while stretching the surrounding arterial wall. Percutaneous transluminal balloon angioplasty treatments require re-intervention due to restenosis in as many as 50% of patients when a plain balloon-tipped catheter is used. Repeated re-intervention can be costly, dangerous, and undesirable for patients. Drug-coated catheter balloons have shown significantly lower restenosis rates, but the drug coatings have been difficult to optimize. Multiple different methods are used to coat catheter balloons with an anti-proliferative drug. These methods include micro-pipetting, solvent casting, and dip coating. However, currently available drug-coated balloons have relatively low drug transfer rates to the desired lesions. A variety of factors can produce these low drug transfer rates including coating solubility, coating mechanical strength, and overall coating stability. Therefore, there exists a need for coated devices that have proper coating solubility, coating mechanical strength, and overall coating stability for use in angioplasty procedures.

### SUMMARY

The instant disclosure is directed to electrospun fiber-coated angioplasty devices and methods. In some embodiments, an angioplasty device may comprise a catheter, a balloon disposed on the catheter, and a scaffold disposed over the balloon. The scaffold may comprise an electrospun polymer fiber and an agent dispersed within the polymer fiber. In some embodiments, the agent may comprise a compound such as an anti-proliferative compound, a vasodilator, a vasoconstrictor, an analgesic, or combinations thereof.

In some embodiments, a method of making such an angioplasty device may comprise obtaining a catheter having a balloon disposed on the catheter, contracting the balloon, and electrospinning a polymer solution onto the surface of the catheter having the balloon, thereby forming a scaffold disposed over the balloon. The polymer solution may comprise a polymer, a solvent, and an agent, and the scaffold may comprise an electrospun polymer fiber and the agent dispersed within the electrospun polymer fiber.

In some embodiments, a method of performing an angioplasty procedure on a subject or patient in need thereof may comprise inserting such an angioplasty device into a blood vessel of the subject, placing the angioplasty device near a lesion within the blood vessel, expanding the balloon of the angioplasty device for a period of time, thereby contacting the lesion with the scaffold, contracting the balloon of the angioplasty device, and removing the angioplasty device from the blood vessel. In some embodiments, the scaffold may contract to maintain contact with a surface of the balloon during the step of contracting the balloon; in other embodiments, the scaffold may maintain contact with the lesion during the step of contracting the balloon.

### BRIEF DESCRIPTION OF THE DRAWINGS

FIG. 1A shows an angioplasty device comprising a catheter, a balloon disposed on the catheter, and a scaffold disposed over the balloon, the balloon in a contracted state, in accordance with the present disclosure.

FIG. 1B shows the angioplasty device of FIG. 1A with the balloon in an expanded state, in accordance with the present disclosure.

FIG. 2A shows an angioplasty device comprising a catheter, a balloon disposed on the catheter, and a scaffold disposed over the balloon, with the balloon in a contracted state, in accordance with the present disclosure.

FIG. 2B shows the angioplasty device of FIG. 2A with the balloon in an expanded state, in accordance with the present disclosure.

FIG. 3A shows an angioplasty device comprising a catheter, a balloon disposed on the catheter, and a scaffold disposed over the balloon, the balloon in a contracted state, in accordance with the present disclosure.

FIG. 3B shows the angioplasty device of FIG. 3A with the balloon in an expanded state, in accordance with the present disclosure.

FIG. 4A shows an angioplasty device comprising a catheter, a balloon disposed on the catheter, and a scaffold disposed over the balloon, with the balloon in an expanded state, in accordance with the present disclosure.

FIG. 4B shows the angioplasty device of FIG. 4A with the balloon in a contracted state and the scaffold maintaining contact with the surface of the balloon, in accordance with the present disclosure.

FIG. 4C shows a magnified view of the scaffold of FIG. 4B maintaining contact with the surface of the balloon in its contracted state, in accordance with the present disclosure.

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of or “consisting of” means that the device or method includes only the elements, steps, or ingredients specifically recited in the particular claimed embodiment or claim.

In embodiments or claims where the term comprising is used as the transition phrase, such embodiments can also be envisioned with replacement of the term “comprising” with the terms “consisting of' or “consisting essentially of.”

As used herein, the term “balloon” means any device or component that can be expanded or contracted, either mechanically or by inflation with a gas or liquid. In some examples, a balloon may include an inflatable balloon that may be expanded with a gas or liquid. In such examples, the balloon may have stretchy, elastic, or compliant characteristics. In other examples, a balloon may be a rigid, inflexible, or non-compliant balloon that can be folded onto itself, much like an accordion.

As used herein, the term “lesion” means any portion of a subject's tissue that is the target of a procedure, treatment, compression, or pressure. In some examples, a lesion may be a stenosis, or narrowed portion, within a blood vessel.

### Electrospinning Fibers

Electrospinning is a method which may be used to process a polymer solution into a fiber. In embodiments wherein the diameter of the resulting fiber is on the nanometer scale, the fiber may be referred to as a nanofiber. Fibers may be formed into a variety of shapes by using a range of receiving surfaces, such as mandrels or collectors. In some embodiments, a flat shape, such as a sheet or sheet-like fiber mold, a fiber scaffold and/or tube, or a tubular lattice, may be formed by using a substantially round or cylindrical mandrel. In certain embodiments, the electrospun fibers may be cut and/or unrolled from the mandrel as a fiber mold to form the sheet. The resulting fiber molds or shapes may be used in many applications, including filters and the like.

Electrospinning methods may involve spinning a fiber from a polymer solution by applying a high DC voltage potential between a polymer injection system and a mandrel. In some embodiments, one or more charges may be applied to one or more components of an electrospinning system. In some embodiments, a charge may be applied to the mandrel, the polymer injection system, or combinations or portions thereof. Without wishing to be bound by theory, as the polymer solution is ejected from the polymer injection system, it is thought to be destabilized due to its exposure to a charge. The destabilized solution may then be attracted to a charged mandrel. As the destabilized solution moves from the polymer injection system to the mandrel, its solvents may evaporate and the polymer may stretch, leaving a long, thin fiber that is deposited onto the mandrel. The polymer solution may form a Taylor cone as it is ejected from the polymer injection system and exposed to a charge.

In certain embodiments, a first polymer solution comprising a first polymer and a second polymer solution comprising a second polymer may each be used in a separate polymer injection system at substantially the same time to produce one or more electrospun fibers comprising the first polymer interspersed with one or more electrospun fibers comprising the second polymer. Such a process may be referred to as “co-spinning” or “co-electrospinning,” and a scaffold produced by such a process may be described as a co-spun or co-electrospun scaffold.

Polymer injection system

A polymer injection system may include any system configured to eject some amount of a polymer solution into an atmosphere to permit the flow of the polymer solution from the injection system to the mandrel. In some embodiments, the polymer injection system may deliver a continuous or linear stream with a controlled volumetric flow rate of a polymer solution to be formed into a fiber. In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with each syringe containing a different polymer solution. In some embodiments, a charge may be applied to the polymer injection system, or to a portion thereof. In some embodiments, a charge may be applied to a needle or needle-like component of the polymer injection system.

In some embodiments, the polymer solution may be ejected from the polymer injection system at a flow rate of less than or equal to about 5 mL/h per needle. In other embodiments, the polymer solution may be ejected from the polymer injection system at a flow rate per needle in a range from about 0.01 mL/h to about 50 mL/h. The flow rate at which the polymer solution is ejected from the polymer injection system per needle may be, in some non-limiting examples, about 0.01 mL/h, about 0.05 mL/h, about 0.1 mL/h, about 0.5 mL/h, about 1 mL/h, about 2 mL/h, about 3 mL/h, about 4 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any range between any two of these values, including endpoints.

As the polymer solution travels from the polymer injection system toward the mandrel, the diameter of the resulting fibers may be in the range of about 100 nm to about 1500 nm. Some non-limiting examples of electrospun fiber diameters may include about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1,000 nm, about 1,050 nm, about 1,100 nm, about 1,150 nm, about 1,200 nm, about 1,250 nm, about 1,300 nm, about 1,350 nm, about 1,400 nm, about 1,450 nm, about 1,500 nm, about 2,000 nm, about 3,000 nm, about 4,000 nm, about 5,000 nm, about 6,000 nm, about 7,000 nm, about 8,000 nm, about 9,000 nm, about 10,000 nm, about 11,000 nm, about 12,000 nm, about 13,000 nm, about 14,000 nm, about 15,000 nm, or any range between any two of these values, including endpoints. In some embodiments, the electrospun fiber diameter may be from about 300 nm to about 1300 nm, about 500 nm to about 15000 nm, about 300 nm to about 10,000 nm, or a value within any of these ranges.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include, for example, non-resorbable polymers, resorbable polymers, natural polymers, or a combination thereof.

In some embodiments, the polymers may include, for example, nylon, nylon 6,6, polycaprolactone, polyethylene oxide terephthalate, polybutylene terephthalate, polyethylene oxide terephthalate-co-polybutylene terephthalate, polyethylene terephthalate, polyurethane, polyethylene, polyethylene oxide, polyvinylpyrrolidone, polyester, polymethylmethacrylate, polyacrylonitrile, silicone, polycarbonate, polylactide, polyglycolide, polyether ketone ketone, polyether ether ketone, polyether imide, polyamide, polystyrene, polyether sulfone, polysulfone, polyvinyl acetate, polytetrafluoroethylene, polyvinylidene fluoride, polylactic acid, polyglycolic acid, polylactide-co-glycolide, polylactide-co-caprolactone, polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, copolymers thereof, and combinations thereof.

It may be understood that polymer solutions may also include a combination of one or more of non-resorbable, resorbable polymers, and naturally occurring polymers in any combination or compositional ratio. In an alternative embodiment, the polymer solutions may include a combination of two or more non-resorbable polymers, two or more resorbable polymers or two or more naturally occurring polymers. In some non-limiting examples, the polymer solution may comprise a weight percent ratio of, for example, from about 5% to about 90%. Non-limiting examples of such weight percent ratios may include about 5%, about 10%, about 15%, about 20%, about 25%, about 30%, about 33%, about 35%, about 40%, about 45%, about 50%, about 55%, about 60%, about 66%, about 70%, about 75%, about 80%, about 85%, about 90%, or ranges between any two of these values, including endpoints.

In some embodiments, the polymer solution may comprise one or more solvents. In some embodiments, the solvent may comprise, for example, polyvinylpyrrolidone, hexafluoro-2-propanol (HFIP), acetone, dimethylformamide, dimethylsulfoxide, N-methylpyrrolidone, N,N-dimethylformamide, Nacetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

In some embodiments, the polymer solution may also include additional materials. Non-limiting examples of such additional materials may include radiation opaque materials, contrast agents, electrically conductive materials, fluorescent materials, luminescent materials, antibiotics, growth factors, vitamins, cytokines, steroids, anti-inflammatory drugs, small molecules, sugars, salts, peptides, proteins, cell factors, DNA, RNA, other materials to aid in non-invasive imaging, or any combination thereof. In some embodiments, the radiation opaque materials may include, for example, barium, tantalum, tungsten, iodine, gadolinium, gold, platinum, bismuth, or bismuth (III) oxide. In some embodiments, the electrically conductive materials may include, for example, gold, silver, iron, or polyaniline.

In certain embodiments, the polymer solution may comprise an agent. The agent may be, for example, any agent that comprises a compound for affecting cellular changes in a tissue. In some embodiments, a compound that affects cellular change includes a compound for affecting the cell growth, phenotype, viability, genes, mitochondria, and the like. In some embodiments, the agent may be a pharmaceutical. In certain embodiments, the agent may be, for example, an anti-proliferative compound, a vasodilator, a vasoconstrictor, an analgesic, or any combination thereof. In some embodiments, the agent may comprise an anti-proliferative compound selected from paclitaxel, sirolimus, zotarolimus, or any combination thereof. In other embodiments, the agent may be selected from miRNA, a gene vector, a peptide, a stem cell, a protein, a ligand, a lipid, or any combination thereof.

In some embodiments, the additional materials and/or agents may be present in the polymer solution or in the resulting electrospun polymer fibers in an amount from about 1 wt % to about 1500 wt % of the polymer mass. In some non-limiting examples, the additional materials may be present in the polymer solution or in the resulting electro spun polymer fibers in an amount of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 100 wt %, about 125 wt %, about 150 wt %, about 175 wt %, about 200 wt %, about 225 wt %, about 250 wt %, about 275 wt %, about 300 wt %, about 325 wt %, about 350 wt %, about 375 wt %, about 400 wt %, about 425 wt %, about 450 wt %, about 475 wt %, about 500 wt %, about 525 wt %, about 550 wt %, about 575 wt %, about 600 wt %, about 625 wt %, about 650 wt %, about 675 wt %, about 700 wt %, about 725 wt %, about 750 wt %, about 775 wt %, about 800 wt %, about 825 wt %, about 850 wt %, about 875 wt %, about 900 wt %, about 925 wt %, about 950 wt %, about 975 wt %, about 1000 wt %, about 1025 wt %, about 1050 wt %, about 1075 wt %, about 1100 wt %, about 1125 wt %, about 1150 wt %, about 1175 wt %, about 1200 wt %, about 1225 wt %, about 1250 wt %, about 1275 wt %, about 1300 wt %, about 1325 wt %, about 1350 wt %, about 1375 wt %, about 1400 wt %, about 1425 wt %, about 1450 wt %, about 1475 wt %, about 1500 wt %, or any range between any of these two values, including endpoints.

The type of polymer in the polymer solution may determine the characteristics of the electrospun fiber. Some fibers may be composed of polymers that are bio-stable and not absorbable or biodegradable when implanted. Such fibers may remain generally chemically unchanged for the length of time in which they remain implanted. Alternatively, fibers may be composed of polymers that may be absorbed or bio-degraded over time, slowly, rapidly, or at any rate in between slowly and rapidly. It may be further understood that a polymer solution and its resulting electrospun fiber(s) may be composed or more than one type of polymer, and that each polymer therein may have a specific characteristic, such as bio-stability, biodegradability, or bioabsorbability.

### Applying Charges to Electrospinning Components

In an electrospinning system, one or more charges may be applied to one or more components, or portions of components, such as, for example, a mandrel or a polymer injection system, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to mandrel, or portions thereof. In some embodiments, a negative charge may be applied to the mandrel, or portions thereof. In some embodiments, the mandrel, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about −15 kV to about 30 kV, including endpoints. In some non-limiting examples, the charge applied to any component of the electrospinning system, or portions thereof, may be about −15 kV, about −10 kV, about −5 kV, about −4 kV, about −3 kV, about −1 kV, about −0.01 kV, about 0.01 kV, about 1 kV, about 5 kV, about 10 kV, about 11 kV, about 11.1 kV, about 12 kV, about 15 kV, about 20 kV, about 25 kV, about 30 kV, or any range between any two of these values, including endpoints. In some embodiments, any component of the electrospinning system, or portions thereof, may be grounded.

Mandrel Movement during Electrospinning

During electrospinning, in some embodiments, the mandrel may move with respect to the polymer injection system. In some embodiments, the polymer injection system may move with respect to the mandrel. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the mandrel may be in contact with or mounted upon a support structure that may be moved using one or more motors or motion control systems. The pattern of the electrospun fiber deposited on the mandrel may depend upon the one or more motions of the mandrel with respect to the polymer injection system. In some embodiments, the mandrel surface may be configured to rotate about its long axis. In one non-limiting example, a mandrel having a rotation rate about its long axis that is faster than a translation rate along a linear axis, may result in a nearly helical deposition of an electrospun fiber, forming windings about the mandrel. In another example, a mandrel having a translation rate along a linear axis that is faster than a rotation rate about a rotational axis, may result in a roughly linear deposition of an electrospun fiber along a liner extent of the mandrel.

### Electrospun Fiber-Coated Angioplasty Devices and Methods

The instant disclosure is directed to electrospun fiber-coated angioplasty devices and methods. It may be understood that the devices and methods described herein may be applied to any medical procedure, and that the examples described herein are non-limiting.

Cardiovascular disease is the leading cause of death among Americans. Atherosclerosis, or the narrowing of a blood vessel due to the buildup of plaque from fat or cholesterol deposition, is frequently treated using percutaneous transluminal angioplasty. During an angioplasty procedure, a balloon-tipped catheter is directed through the blood vessel toward the site of the constriction. After being positioned inside the stenosis, the balloon is dilated for a precise amount of time prior to removing the catheter. This dilation breaks apart and compresses the plaque buildup while stretching the surrounding vessel wall. Additionally, balloon-tipped catheters can be used to treat in-stent restenosis that may occur following prior angioplasty procedures.

Percutaneous transluminal balloon angioplasty treatments require re-intervention due to restenosis in as many as 50% of patients in whom a plain balloon is initially used. Implanted stents produce a similarly high rate of re-intervention due to in-stent stenosis. Repeated re-intervention can be costly, dangerous, and undesirable for patients. Drug-coated catheter balloons have recently emerged as new technology for the treatment of peripheral artery disease, in-stent restenosis, and small vessel disease. These drug-coated balloons, used in percutaneous transluminal balloon angioplasty procedures, show significantly lower percentages of patients needing re-intervention due to restenosis when compared to the plain balloon catheter treatment data. At various time points post-procedure, drug-coated balloons have shown lower rates of late lumen loss and lower rates of binary restenosis when compared to a plain balloon catheter.

Multiple different methods are used to coat catheter balloons with an anti-proliferative drug. These methods include micro-pipetting, solvent casting, and dip coating. Regardless of the procedure by which they are made, all of the currently available drug-coated balloons have relatively low drug transfer rates to the desired lesions. A variety of factors can produce these low drug transfer rates including coating solubility, coating mechanical strength, and overall coating stability.

The mechanical strength of the coating in particular presents a special challenge for the creation of a drug coating. Coatings on the exterior of the balloon must have sufficient mechanical strength to prevent the coating and the drug from being lost during movement of the catheter throughout the angioplasty procedure, while simultaneously deficient to the mechanical forces caused by the dilation of the balloon during the procedure designed to induce drug transfer to the lesion. Another factor to consider regarding mechanical strength of the drug coating is ex vivo pre-dilation of the catheter balloon, and the need for sufficient mechanical strength to prevent the coating from being disrupted during this step. Optimizing this mechanical strength range for peak drug delivery to the desired area of stenosis while considering the needed coating solubility and stability suggests the need for a new category of drug-coated angioplasty devices with higher efficacy in drug delivery.

In addition to optimizing the mechanical strength range of the coating, it is advantageous to use a solution for the drug coating with a solubility that allows for quick release of the drug, but not so quick that a large portion of the drug is lost during the movement of the balloon catheter through the vessel toward the location of the stenosis. Typically, the catheter balloon is expanded or inflated for a relatively short period of time, ranging from about one minute to about five minutes, once it reaches the lesion. This time frame presents a need for a precise solubility of the coating to allow for optimal drug delivery during balloon dilation in vivo.

Electrospinning a polymer solution onto a catheter balloon, as described herein, to produce an electrospun fiber-coated angioplasty device, is an optimal method for refining important qualities of the balloon coating. Incorporating an agent, such as an anti-proliferative drug, into a polymer solution that can be electrospun onto a catheter balloon for use in percutaneous transluminal balloon angioplasty has the promising ability to increase drug delivery rates for drug-coated balloon catheters. Multiple polymers could be considered for the solution used to incorporate the drug, as described herein. Altering the polymer solution used in production may affect the material properties, solubility, and stability of the coating.

Using electrospun polymer fibers to create the coating on the balloon allows for precise control of the degradation time and thus the time of release of the agent or drug. In some embodiments, for example, polyethylene oxide and/or polyvinylpyrrolidone, for example, are polymers with rapid degradation timeframes, and thus are important candidates to consider for the solubility of the coating and the time frame in which the agent must be delivered.

Additionally, incorporating the agent into the fibers of the polymer may allow for more control of the agent delivery, because the incorporation may ensure that the agent is not lost before the catheter balloon arrives at the site of the lesion and is expanded or dilated. This is a key factor in achieving the desired release of the agent to the lesion. Others have attempted to incorporate electrospun polymer fibers into angioplasty devices, but have failed to incorporate the agent into the electrospun polymer fibers themselves. Korean Patent Publication No. 10-2018-0012885, for example, attempts to prevent the premature release of a drug coating on an angioplasty device by applying a protective layer of electrospun fibers over the drug coating. The two distinct layers (a drug layer and a fiber layer) are clearly shown in the publication's figures. Unlike the instant disclosure, however, Korean Patent Publication No. 10-2018-0012885 fails to incorporate the agent (or drug) into the electrospun fibers themselves.

Incorporating agents into electrospun polymer fibers for catheter balloon coatings has several advantages. For example, doing so may help increase the consistency of even, or substantially or largely even, controlled spacing during agent delivery, rather than delivering the agent in inconsistent dosages to different areas of the vessel tissue. This improved consistency or uniformity ensures that a maximum amount of the target lesion or tissue receives the incorporated agent at a consistent dosage, allowing for a more effective percutaneous transluminal balloon angioplasty procedure as a whole and thus a lower rate of restenosis and a reduced need for re-intervention.

Certain polymers may also produce high levels of elasticity in electrospun polymer fibers, allowing for sufficient mechanical strength to withstand the pre-dilation of the catheter balloon and the friction forces exerted upon the balloon and its coating while being guided through the vessel towards the lesion. Tailoring the polymer and agent solution used in electrospinning may lend the capability to produce a coating with mechanical properties that fall within the optimal or preferred range of values to deliver the agent while withstanding friction and radial forces.

In some embodiments, an angioplasty device may comprise a catheter and a balloon disposed on the catheter. The catheter may include any catheter capable of use in a medical procedure. In some embodiments, the balloon may be disposed on one end of the catheter. In certain embodiments, the balloon may be disposed on the proximal end of the catheter. As described herein, the balloon may comprise any device or component that can be expanded or contracted, either mechanically or by inflation with a gas or liquid. In some examples, a balloon may include an inflatable balloon that may be expanded with a gas or liquid. In such examples, the balloon may have stretchy, elastic, or compliant characteristics. In other examples, a balloon may be a rigid, inflexible, or non-compliant balloon that can be folded onto itself, much like an accordion.

In some embodiments, the angioplasty device may further comprise a scaffold disposed over the balloon. The scaffold may be disposed over the balloon such that it contacts a portion, or in some cases a significant portion, of the balloon when it is expanded and/or contracted, in some non-limiting embodiments. In some embodiments, the scaffold may comprise one or more electrospun polymer fibers as described herein. In certain embodiments, the scaffold may further comprise an agent dispersed within the electrospun polymer fiber. Such a fiber may be formed by the methods described herein, including by including the polymer and the agent in the polymer solution used to electrospun the polymer fiber. This process may, in some embodiments, result in the agent being truly dispersed, well-dispersed, or substantially dispersed within the electrospun polymer fiber, rather than the electrospun polymer fiber being coated with the agent or the agent being applied to the electrospun polymer fiber after the fiber is formed.

In some embodiments, the agent may comprise any agent or additive described herein. The agent may comprise, for example, a compound for affecting cellular changes. In some embodiments, a compound that affects cellular changes includes a compound for affecting the cell growth, phenotype, viability, genes, mitochondria, and the like. In some embodiments, the agent may include, for example, an anti-proliferative compound, a vasodilator, a vasoconstrictor, an analgesic, a derivative thereof, an analogue thereof, or any combination thereof. In some embodiments, an anti-proliferative compound may include, for example, paclitaxel, sirolimus, zotarolimus, a derivative thereof, an analogue thereof, or any combination thereof. In still other embodiments, the agent may include, for example, miRNA, a gene vector, a peptide, a stem cell, a protein, a ligand, a lipid, a derivative thereof, an analogue thereof, or any combination thereof.

In certain embodiments, the electrospun polymer fiber may comprise any polymer described herein, including for example, polyethylene oxide, polyvinylpyrrolidone, polyurethane, polylactide, polyglycolide, any copolymer thereof, or any combination thereof. In some embodiments, the electrospun polymer fiber may comprise a polymer that will rapidly degrade upon exposure to a tissue. Non-limiting examples of such rapidly degrading polymers may include polyethylene oxide and polyvinylpyrrolidone. In other embodiments, the electrospun polymer fiber may comprise a polymer that will degrade more slowly upon exposure to a tissue. Non-limiting examples of such slow degrading polymers may include polyglycolide, polylactide, or co-polymers of those two. In some embodiments, for example, the electrospun polymer may comprise a rapidly degrading polymer that will degrade in a period of time from about 15 seconds to about 7 minutes upon exposure to the tissue. In some embodiments, for example, the electrospun polymer may comprise a rapidly degrading polymer that will degrade in a period of time from about 1 day to about 180 days upon exposure to the tissue. The period of degradation time may be, for example, about 15 seconds, about 30 seconds, about 45 seconds, about 1 minute, about 1.5 minutes, about 2 minutes, about 2.5 minutes, about 3 minutes, about 3.5 minutes, about 4 minutes, about 4.5 minutes, about 5 minutes, about 5.5 minutes, about 6 minutes, about 6.5 minutes, about 7 minutes, about 30 minutes, about 1 hour, about 3 hours, about 5 hours, about 7 hours, about 10 hours, about 12 hours, about 15 hours, about 24 hours, about 2 days, about 5 days, about 10 days, about 15 days, about 30 days, about 45 days, about 60 days, about 75 days, about 90 days, about 120 days, about 180 days, or any range between any two of these values, including endpoints. In certain embodiments, the tissue may comprise a lesion, while in other embodiments the tissue may comprise the tissue surrounding the lesion, tissue elsewhere in the vessel, or any other tissue of the subject or patient.

In some embodiments, the scaffold may be configured to stretch or relax in order to maintain contact with the surface of the balloon when the balloon is expanded. In such embodiments, the scaffold may be capable of such stretching or relaxation without disrupting the integrity of the scaffold. In other embodiments, the scaffold may be configured to contract, relax, or shrink in order to maintain contact with the surface of the balloon when the balloon is contracted. In some embodiments, the scaffold may maintain contact with the surface of the balloon when the balloon is contracted after it has been expanded. In certain embodiments, these features may allow the scaffold to maintain its structural integrity and the integrity of any agents dispersed within the fiber or fibers of the scaffold as the balloon is expanded and contracted, even over multiple cycles of expansion and/or contraction of the balloon.

In some embodiments, a method of making the angioplasty devices described herein may include any electrospinning methods described herein. The method of making an angioplasty device may comprise obtaining a catheter having a balloon disposed on the catheter, as described herein. In some embodiments, the method may further comprise contracting the balloon before proceeding with the step of electrospinning; in other embodiments, the method may further comprise expanding the balloon before proceeding with the step of electrospinning.

In some embodiments, the method may further comprise electrospinning a polymer solution onto the surface of the catheter having the balloon, as described herein, thereby forming a scaffold disposed over the balloon, as described herein. In certain embodiments, the polymer solution may comprise a polymer, a solvent, and an agent, as described herein. In some embodiments, the scaffold may comprise an electrospun polymer fiber and the agent dispersed within the electrospun polymer fiber, as described herein.

In some embodiments, methods of performing angioplasty procedures on subjects in need thereof may include using the angioplasty devices described herein. A method of performing an angioplasty procedure on a subject may comprise inserting into a blood vessel of the subject an angioplasty device as described herein. In some embodiments, the method may further comprise advancing the angioplasty device toward a lesion or other target area within the vessel. In certain embodiments, the step of advancing the angioplasty device may not include the substantial degradation of the scaffold, or the delivery of the agent to the tissue. In some embodiments, the method may further comprise placing the angioplasty device near a lesion or other target area within the blood vessel.

In certain embodiments, the method may further comprise expanding the balloon of the angioplasty device for a period of time, thereby contacting the lesion or other target area with the scaffold. In some embodiments, the scaffold may stretch or relax to maintain contact with a surface of the balloon during the step of expanding the balloon. As described herein, the electrospun polymer fiber of the scaffold may comprise a polymer that begins to degrade when the polymer contacts the lesion or other target area. In certain embodiments, the step of contacting the lesion or other target area with the scaffold may comprise delivering the agent to a portion of the lesion or other target area. In such embodiments, the agent may delivered when the electrospun polymer fiber degrades, thereby releasing the agent and delivering it to the cells or tissue. In some embodiments, the electrospun polymer fiber may degrade (and thus, the agent may be released), over a period of time from about 15 seconds to about 7 minutes, as described herein.

In some embodiments, the method may further comprise contracting the balloon of the angioplasty device. In some embodiments, the scaffold may contract or relax to maintain contact with a surface of the balloon during the step of contracting the balloon, as described herein. In alternative embodiments, the scaffold may maintain contact with the lesion or other target area of the vessel during the step of contracting the balloon, such that the scaffold is left at or near the target area or lesion after the catheter is removed. In such embodiments, the electrospun polymer fiber of the scaffold may comprise a more slowly degrading polymer and thereby release the agent more slowly, as described herein.

In some embodiments, the method may further comprise removing the angioplasty device from the blood vessel. In certain embodiments, the scaffold may maintain contact with the surface of the balloon during the step of removing the angioplasty device from the blood vessel. In other embodiments, the scaffold may maintain contact with the lesion or other target area during the step of removing the angioplasty device from the blood vessel. In some embodiments, all of the scaffold would remain in contact with the vessel wall and none would remain on the balloon. In some embodiments, a majority of the scaffold would remain in contact with the vessel wall.

### EXAMPLES

Several prototypes of the angioplasty devices described herein have been made, employing various balloons and scaffolds with electrospun polymer fibers electrospun from various polymer solutions. The various polymer solutions included (i) 10 weight percent poly(ethylene oxide) (PEO) in dichloromethane (DCM); (ii) 10 weight percent polyvinylpyrrolidone (PVP) in ethanol (EtOH); (iii) 3 weight percent polyurethane (PU); and (iv) 10 weight percent PVP.

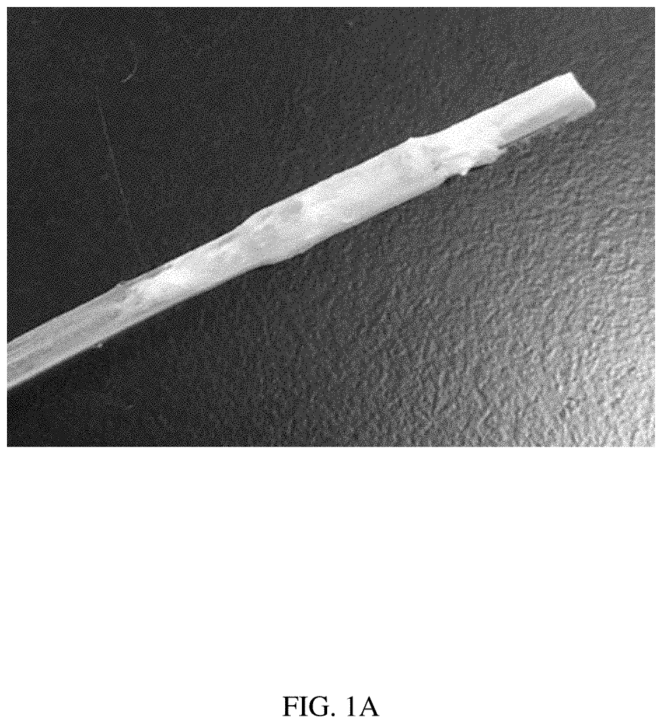

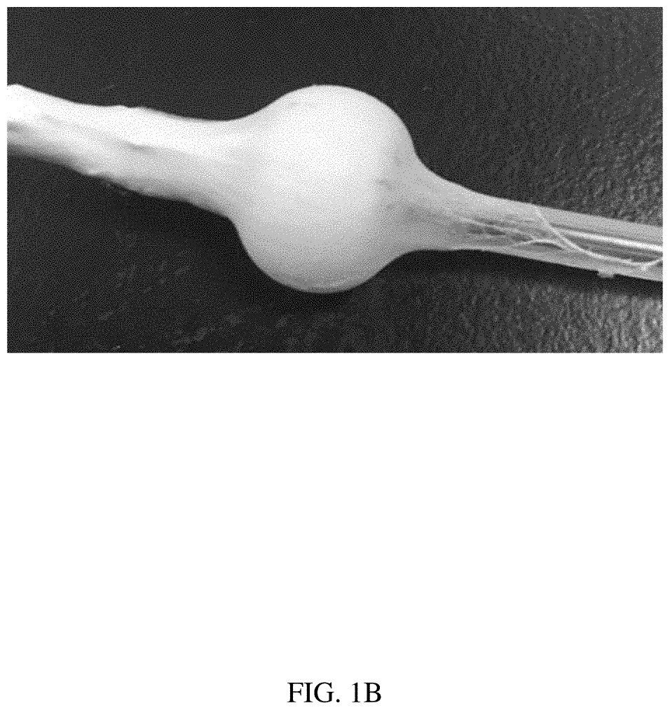

For example, FIG. 1A shows an angioplasty device comprising a catheter, a balloon disposed on the catheter, and a scaffold disposed over the balloon, the balloon in a contracted state. The scaffold of FIG. 1A comprises polymer fibers electrospun from a polymer solution comprising 10 wt % PEO+DCM. FIG. 1B shows the angioplasty device of FIG. 1A with the balloon in an expanded state.

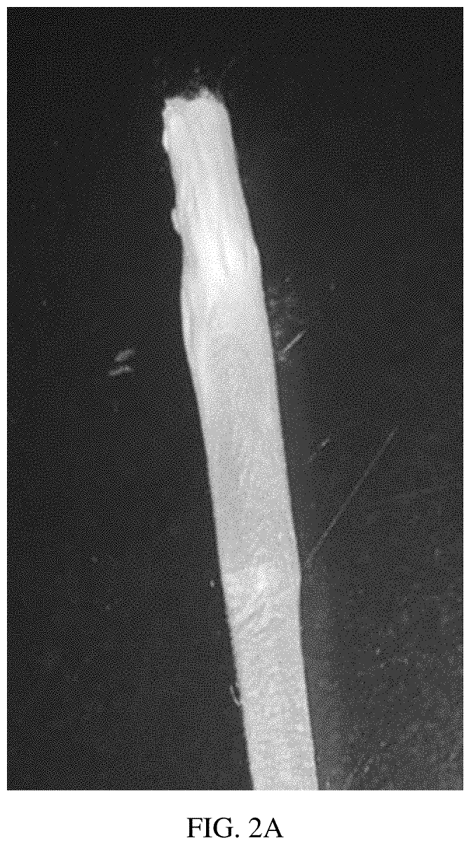

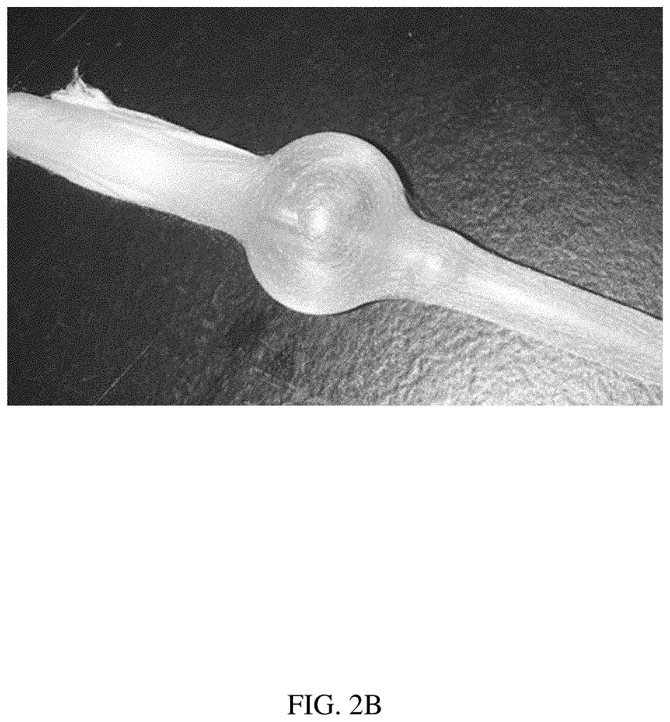

In another example, FIG. 2A shows an angioplasty device comprising a catheter, a balloon disposed on the catheter, and a scaffold disposed over the balloon, the balloon in a contracted state. The scaffold of FIG. 2A comprises polymer fibers electrospun from a polymer solution comprising 10 wt % PVP+EtOH. FIG. 2B shows the angioplasty device of FIG. 2A with the balloon in an expanded state.

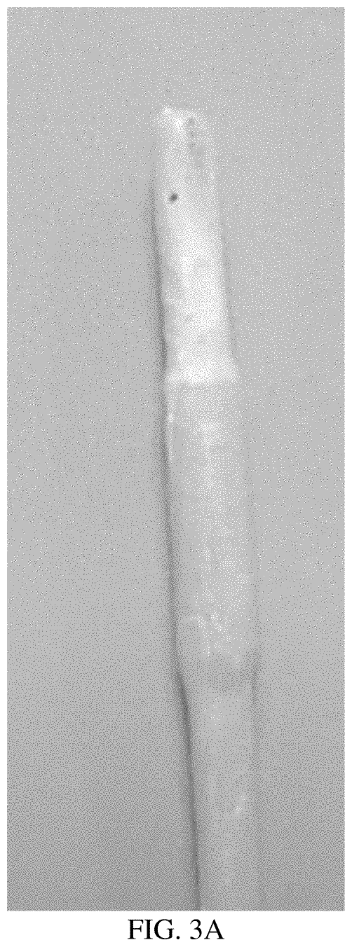

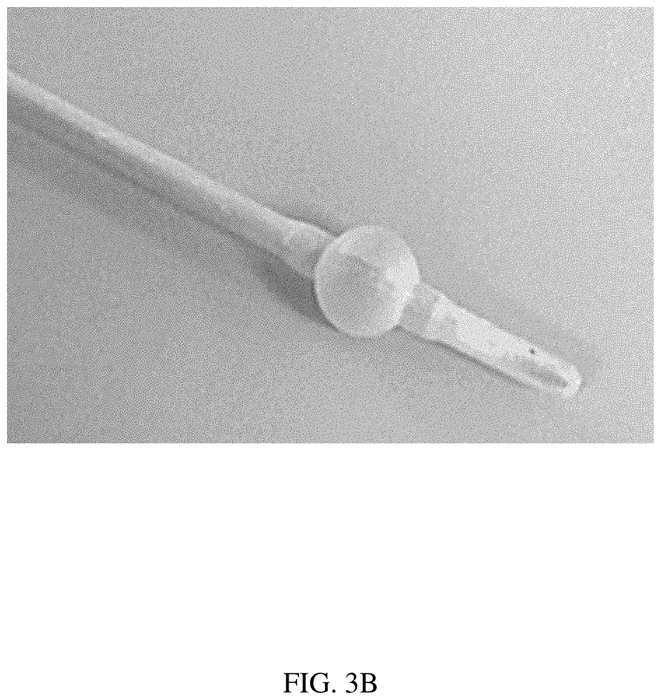

In yet another example, FIG. 3A shows an angioplasty device comprising a catheter, a balloon disposed on the catheter, and a scaffold disposed over the balloon, the balloon in a contracted state. The scaffold of FIG. 3A comprises polymer fibers electrospun from a polymer solution comprising 3 wt % PU. FIG. 3B shows the angioplasty device of FIG. 3A with the balloon in an expanded state.

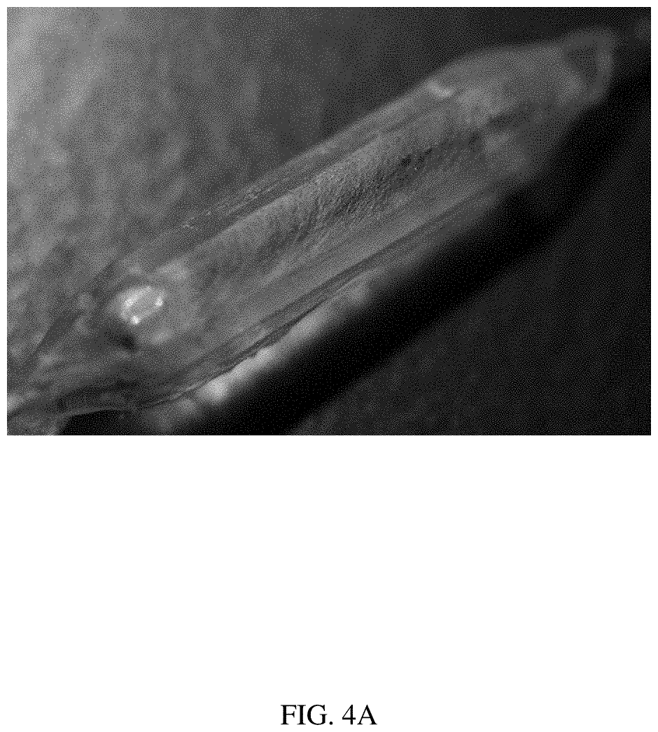

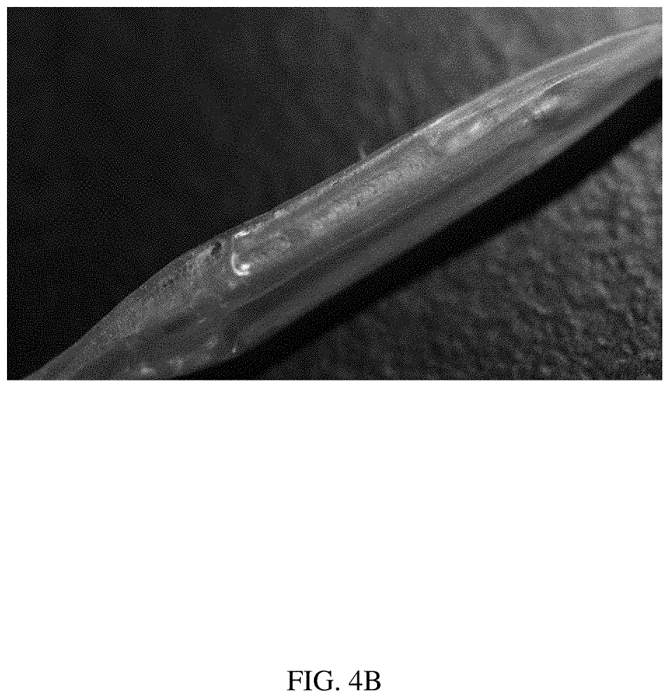

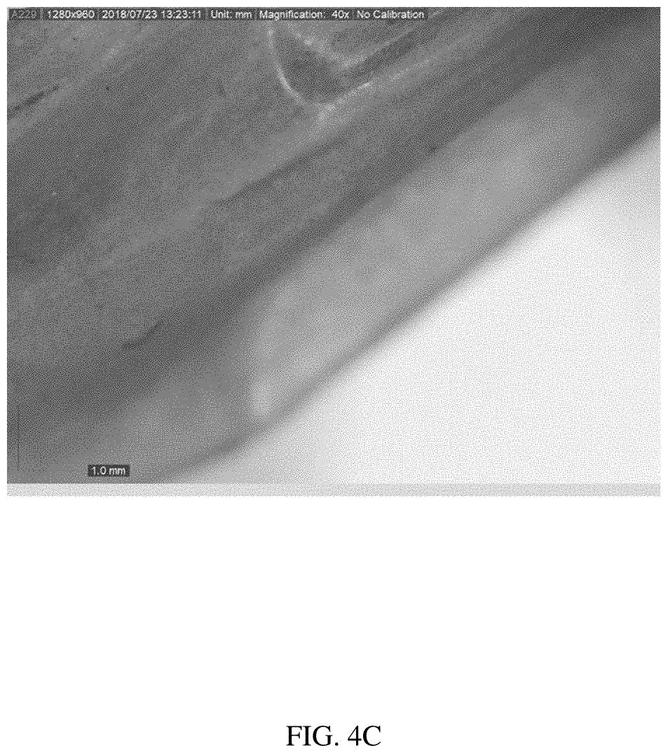

Finally, FIG. 4A shows an angioplasty device comprising a catheter, a balloon disposed on the catheter, and a scaffold disposed over the balloon, the balloon in an expanded state. The scaffold of FIG. 4A comprises polymer fibers electrospun from a polymer solution comprising 10 wt % PVP. FIG. 4B shows the angioplasty device of FIG. 4A with the balloon in a contracted state. The scaffold of FIG. 4B is shown maintaining intimate contact with at least a portion of the surface of the balloon after it is contracted. FIG. 4C shows a magnified view of the scaffold of FIG. 4B maintaining contact with at least a portion of the surface of the balloon in its contracted state.

While the present disclosure has been illustrated by the description of exemplary embodiments thereof, and while the embodiments have been described in certain detail, it is not the intention of the Applicant to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1.-37. (canceled)

38. An angioplasty device comprising:
a catheter;
a balloon disposed on the catheter; and
a scaffold disposed over the balloon;
wherein the scaffold comprises an electrospun polymer fiber and an agent dispersed within the electrospun polymer fiber.

39. The angioplasty device of claim 38, wherein the agent is selected from the group consisting of an anti-proliferative compound, a vasodilator, a vasoconstrictor, an analgesic, and combinations thereof.

40. The angioplasty device of claim 38, wherein the agent comprises an anti-proliferative compound selected from the group consisting of paclitaxel, sirolimus, zotarolimus, and combinations thereof.

41. The angioplasty device of claim 38, wherein the agent is selected from the group consisting of miRNA, a gene vector, a peptide, a stem cell, a protein, a ligand, a lipid, and combinations thereof.

42. The angioplasty device of claim 38, wherein the electrospun polymer fiber comprises a polymer configured to degrade in a time from about 1 minute to about 5 minutes upon exposure to a tissue.

43. The device of claim 42, wherein the tissue comprises a lesion.

44. The angioplasty device of claim 38, wherein the electrospun polymer fiber comprises a polymer selected from the group consisting of polyethylene oxide, polyvinylpyrrolidone, polyurethane, polylactide, polyglycolide, polycaprolactone, polydioxanone, and combinations thereof.

45. The angioplasty device of claim 38, wherein the agent is dispersed substantially homogeneously within the electrospun polymer fiber.

46. The angioplasty device of claim 38, wherein the scaffold is configured to stretch to maintain contact with a surface of the balloon when the balloon is expanded, and wherein the scaffold is configured to contract to maintain contact with the surface of the balloon when the balloon is contracted.

47. A method of making an angioplasty device, the method comprising:
obtaining a catheter having a balloon disposed on the catheter;
contracting the balloon;
electrospinning a polymer solution onto the surface of the catheter having the balloon, thereby forming a scaffold disposed over the balloon;
wherein the polymer solution comprises a polymer, a solvent, and an agent; and
wherein the scaffold comprises an electrospun polymer fiber and the agent dispersed within the electrospun polymer fiber.

48. The method of claim 47, wherein the agent is dispersed substantially homogeneously within the electrospun polymer fiber.

49. A method of performing an angioplasty procedure on a subject in need thereof, the method comprising:
inserting into a blood vessel of the subject an angioplasty device comprising:
a catheter;
a balloon device disposed on the catheter; and
a scaffold disposed over the balloon device;
wherein the scaffold comprises an electrospun polymer fiber and an agent dispersed within the electrospun polymer fiber;
placing the angioplasty device near a lesion within the blood vessel;
expanding the balloon of the angioplasty device for a period of time, thereby contacting the lesion with the scaffold;
contracting the balloon of the angioplasty device; and
removing the angioplasty device from the blood vessel.

50. The method of claim 49, wherein the electrospun polymer fiber comprises a polymer that begins to degrade when the lesion is contacted with the scaffold.

51. The method of claim 49, wherein the step of contacting the lesion with the scaffold comprises delivering the agent to a portion of the lesion.

52. The method of claim 49, wherein the period of time is from about 30 seconds to about 5 minutes.

53. The method of claim 49, wherein the scaffold stretches to maintain contact with a surface of the balloon during the step of expanding the balloon.

54. The method of claim 49, wherein the scaffold contracts to maintain contact with a surface of the balloon during the step of contracting the balloon.

55. The method of claim 49, wherein the scaffold maintains contact with a surface of the balloon during the step of removing the angioplasty device from the blood vessel.

56. The method of claim 49, wherein the scaffold maintains contact with the lesion during the step of contracting the balloon.

57. The method of claim 49, wherein the scaffold maintains contact with the lesion during the step of removing the angioplasty device from the blood vessel.
